(19) United States
(12) Patent Application Publication
(10) Pub. No.: US 2001/0031753 A1
(43) Pub. Date: Oct. 18, 2001

(54) Process for the preparation of substituted benzophenones

(75) Inventor: Venkataraman Kameswaran, Pennington, NJ (US)

Correspondence Address: Intellectual Property Department, BASF Corporation, 3000 Continental Drive - North, Mount Olive, NJ, 07828-1234, US

(73) Assignee: BASF Corporation, Mount Olive, NJ (US)

(21) Appl. No.: 09/758,809

(22) Filed: Jan. 11, 2001

Related U.S. Application Data
(60) Provisional application No. 60/175,979, filed on Jan. 13, 2000.

(51) Int. Cl. A61K 31/535; A01N 33/18; A01N 33/24

(52) U.S. Cl. 514/237.5; 514/676

(57) ABSTRACT

There is provided an efficient and effective process for the preparation of a compound of formula I (I)

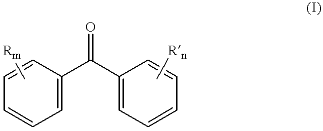

via the acylation of the appropriate substituted benzene substrate in the presence of graphite, optionally in the presence of FeCl₃.

Compounds of formula I are useful as intermediates in the manufacture of agrichemical agents.

### BACKGROUND OF THE INVENTION

Benzophenone compounds are key intermediates for highly active fungicidal and herbicidal agents. In particular, U.S. Pat. No. 4,912,217 describes the use of benzophenones as intermediates in the preparation of 3,3-diphenylacrylic acid amide fungicides. Said fungicides are especially useful for the control of phytopathogenic fungi such as phyophthora in potatoes, tomatoes and strawberries. In addition, the use of certain benzophenone compounds as fungicidal agents has been disclosed in EP 0897904A. Said benzophenone fungicides control ascomycetes in cereals, cucumber, apple or grape. Further, U.S. Pat. No. 5,484,763 and U.S. Pat. No. 5,523,278 describe the use of benzophenones as intermediates in the preparation of benzisoxazole and benzisothiazole herbicidal compounds. In particular, said herbicidal compounds are highly active against both broadleaf and grass species at low use rates, and demonstrate selectivity in soybean, and cereal crops. Said compounds also have utility as broad spectrum postemergent herbicides in low-till and no-till agronomic practice. Said acid amide, benzophenone, benzisoxazole and benzisothiazole crop protection agents are useful for a variety of integrated pest management programs. These programs are essential in today's crop production. Therefore, the manufacture of these herbicidal and fungicidal agents in an economic environmentally safe, and ecologically sound manner is highly desirable.

Friedel-Crafts reactions (see Friedel Crafts and Related Reactions, G. Olah ed., Wiley-Interscience, New York, 1964) have been the primary means to prepare benzophenones. However, said reaction requires the use of a strong Lewis Acid catalyst, especially aluminum chloride. Said catalysts generate large quantities of acid and toxic byproducts, which require neutralization and dilution prior to disposal. Such catalysts cannot be recycled, which adds to the cost of manufacture. A number of Friedel-Crafts catalysts other than aluminum chloride are also known, but most have similar drawbacks regarding the isolation of final product and recycling of catalyst. Recent publications have disclosed the use of graphite alone as a catalyst for a specific limited set of Friedel-Crafts acylations (M. Kodomari, J. Chem. Soc., Chem. Commun., 1997, 1567; Chemistry Letters, 1998, 319; Poster presentation, ICOS 12, Venice, July 1998 #PA 137). These publications disclose conditions which require benzene or chlorobenzene as solvent and significantly high loadings of graphite. Further, the published procedures are applicable to a limited variety of substituted benzophenone products and do not provide for ready access to the substituted benzophenones required for the manufacture of useful agrichemicals and agrichemical intermediates.

Therefore, it is an object of the present invention to provide an efficient preparation of a wide variety of substituted benzophenones with relatively low loadings of graphite or graphite/ferric chloride catalysts.

It is another object of this invention to provide an economic and environmentally safe source of a wide variety of commercially useful benzophenone intermediates and agrichemical products.

It is a feature of this invention that commercial production of benzophenone agrichemical agents and intermediates therefor may be accomplished without the requisite use of an acid chloride reagent.

Other objects and features of the invention will become more apparent from the detailed description set forth hereinbelow.

### SUMMARY OF THE INVENTION

The present invention provides a process for the preparation of a benzophenone compound of formula I

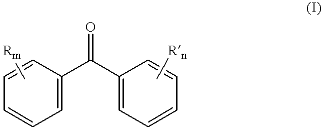

wherein m and n are each independently 0 or an integer of 1, 2, 3, 4 or 5;

R is halogen, C₁-C₆alkyl, C₁-C₆haloalkyl, C₁-C₆alkoxy, C₁-C₆alkoxyalkyl, CO₂R₁, S(O)ₚR2, NR₃R₄, NO₂ or CN;

R′ is C₁-C₆alkyl, C₁-C₆alkoxy, C₁-C₆alkoxyalkyl, or NR₅R₆;

R₁, R₂, R₃, R₄, R₅ and R₆ are each independently C₁-C₆alkyl; and

p is 0 or an integer of 1 or 2

which process comprises reacting a compound of formula II

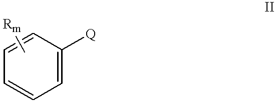

wherein Q is CX₃ or COX; X is Cl or Br; and R and m are as described hereinabove with at least one molar equivalent of a compound of formula III

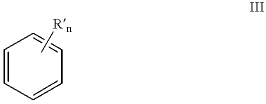

wherein R′ and n are as described hereinabove in the presence of graphite and an inert solvent, optionally in the presence of FeC1₃, and when Q is CX₃ in the presence of at least one molar equivalent of water.

Also provided is the use of the formula I benzophenone compound in the commercial manufacture of an agrichemical agent.

### DETAILED DESCRIPTION OF THE INVENTION

Processes, to be useful on a manufacturing scale, preferentially contain key intermediate compounds which may be obtained in high to quantitative yield, which are stable either upon isolation or in situ and which may be produced from simple or readily available starting materials. Further, the most useful key intermediate compounds are those compounds which may be readily converted to the desired end product of manufacture in a minimum of reaction steps and with a minimum of undesirable side products in optimum yield and purity and, if applicable, regio- or stereospecifically.

Surprisingly, it has now been found that key benzophenone compounds of formula I may be prepared efficiently and effectively via the acylation of an appropriate benzene substrate of formula III in the presence of graphite, optionally in the presence of FeCl₃. The compounds of formula I are highly useful as fungicidal agents and as key intermediates in the manufacture of agrichemicals, such as 3,3-diphenylacrylic acid amide fungicides or benzisoxazole and benzisothiazole herbicides.

Advantageously, the process of the invention reduces the use of graphite or graphite/ferric chloride catalysts, eliminates the need for aluminum chloride catalysts and offers an alternative to the use of acid chloride starting materials in the manufacture of key benzophenone compounds of formula I.

In accordance with the process of the invention, a compound of formula III

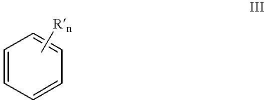

wherein n is 0 or an integer of 1, 2, 3, 4 or 5;

R′ is C₁-C₆alkyl, C₁-C₆alkoxy, C₁-C₆alkoxyalkyl, or NR₅R₆; and

R₅ and R₆ are each independently C₁-C₆alkyl

is acylated with a compound of formula II

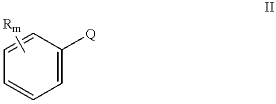

wherein m is 0 or an integer of 1, 2, 3, 4 or 5;

R is halogen, C₁-C₆alkyl, C₁-C₆haloalkyl, C₁-C₆-alkoxy, C₁-C₆alkoxyalkyl, CO₂R1, S(O)ₚR2, NR₃R₄, NO₂ or CN;

Q is CX3 or COX;

X is Cl or Br;

R₁, R₂, R₃ and R₄ are each independently C₁-C6alkyl;and

p is 0 or an integer of 1 or 2

in the presence of graphite and an inert solvent, optionally in the presence of FeCl₃, and when Q is CX₃ in the presence of at least one molar equivalent of water. The reaction sequence is shown in Flow Diagram I wherein the term catalyst designates graphite or a combination of graphite and FeCl₃.

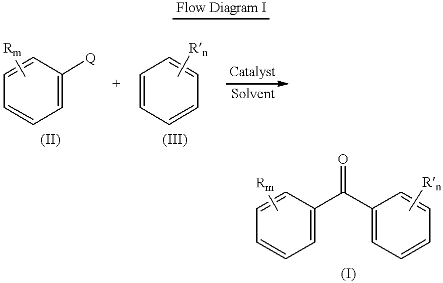

The term halogen as used in the specification and claims designates Cl, Br, F or I. The term haloalkyl designates and alkyl group CₙH₂ₙ₊₁ which is substituted with from 1 to 2n+1 halogen atoms which may be the same or different.

Solvents suitable for use in the process of the invention include halogenated aliphatic hydrocarbons such as dichloroethane, trichloroethane, tetrachloroethane or the like; aromatic hydrocarbons having one or more electron-withdrawing groups such as halobenzene, nitrobenzene, or the like; ethers such as dioxane, tetrahydrofuran, ethylene glycol or the like; or any conventional inert solvent which is incapable of participating in the reaction process, preferably halogenated aliphatic hydrocarbons or halogenated aromatic hydrocarbons, more preferably halogenated aliphatic hydrocarbons.

In the process of the invention the reaction rate is directly related to the reaction temperature, i.e. increased reaction temperatures lead to increased reaction rate. However, excessively high reaction temperatures may lead to an increase of undesired side products and decreased product yield and purity. In general, suitable reaction temperatures may range from about 25° C. to 200° C., preferably about 50° C. to 180° C.

Formula II compounds wherein Q is CX₃ are commercially available or may be obtained by conventional means, such as halogenation of the corresponding substituted toluene precursor with halogenating agents such as chlorine, bromine, N-bromosuccinamide, or N-chlorosuccinimide.

Similarly, formula II compounds wherein Q is COX are available commercially or may be obtained by conventional procedures such as hydrolysis of the corresponding benzotrihalide precursor, or by the reaction of the corresponding carboxylic acid precursor with oxychloride, phosphorous pentachloride, phosgene, thionyl chloride, or any of the conventional reagents used to convert a substituted benzoic acid to the corresponding benzoyl halide.

In actual practice, a compound of formula II is admixed with a compound of formula III (substrate) in the presence of an inert solvent, preferably a halogenated aliphatic hydrocarbon or a halogenated aromatic hydrocarbon, more preferably a halogenated aliphatic hydrocarbon, and graphite preferably about 10 g/mole of substrate to 200 g/mole of substrate, more preferably about 25 g/mole to 100 g/mole, optionally in the presence of FeCL₃, preferably about 0.001 mole % to 1.0 mole %, more preferably about 0.05 mole % to 0.5 mole % and when the formula II compound is a compound wherein Q is CX₃, in the presence of at least one molar equivalent of water, preferably about 1 molar equivalent to 3 molar equivalents, at a temperature of about room temperature to the boiling point of the solvent, preferably about 25° C. to 200° C., more preferably about 50C to 180° C., to form the desired formula I benzophenone. The formula I product may be isolated using conventional methods such as filtration, extraction, chromatography or the like.

Advantageously, the process of the invention employs reaction conditions which are surprisingly mild, require a very low loading of catalyst and provide formula I benzophenone products in relatively high yield and purity with essentially no toxic waste products, as compared to the standard Friedel-Crafts reaction conditions. Surprisingly, the process of the invention utilizes formula II compounds wherein Q is CX₃ to prepare benzophenone formula I compounds and thereby, offers an alternative to the use of an acid chloride reagent to accomplish an effective and efficient acylation procedure.

Compounds of formula I are useful as fungicidal agents and as key intermediates in commercial chemical production, particularly agrichemical production. Accordingly, in one embodiment of the invention the benzophenone compound of formula I prepared from the compounds of formula II and III as described hereinabove, may be reacted with N-acetylmorpholine in the presence of a sodium tert-alkoxide, optionally in the presence of a solvent, to give the fungicidal compound of formula IV. The reaction is illustrated in flow diagram II.

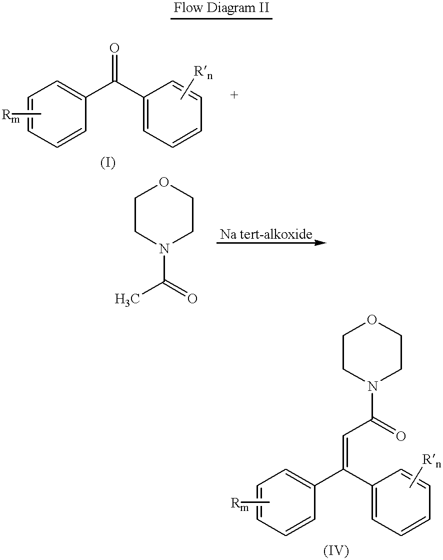

Reactions of substituted benzophenones to form diphenyl acrylic acid amide fungicides of formula IV are described in EP 897,904.

In order to provide a more clear understanding of the invention, the following examples are set forth below. The examples are merely illustrative and are not to be understood to limit the scope or underlying principles of the invention in any way.

The terms ¹HNMR and ¹³CNMR designate proton and carbon 13 NMR respectively. The terms HPLC, TLC and GLC designate high performance liquid chromatography, thin layer chromatography and gas-liquid chromatography, respectively. The term MS designates mass spectrum. Unless otherwise stated, all parts are parts by weight.

### EXAMPLE 1

Preparation of 3′-Bromo-2,3,4,6′-tetramethoxy-2′, 6-dimethylbenzophenone

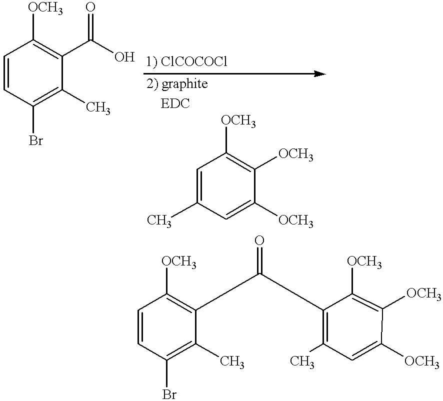

A slurry of 3-bromo-6-methoxy-2-methylbenzoic acid, (4.9 g, 0.02 mol) in 1,2-dichloroethane (EDC) is treated with oxalyl chloride (2.7 g, 0.022 mol) at room temperature over a 15 minute period, heated to 60° C. for 30 minutes, cooled to room temperature, treated with 3,4,5-trimethoxytoluene (2.73 g, 0.015 mol) and graphite (1.0 g), heated at reflux temperature for 2.5 hours [reaction is complete by TLC (silica gel, 25t ethyl acetate/heptane)], cooled to room temperature, and filtered. The filtrate is washed with saturated NaHCO₃ solution and concentrated in vacuo to give a residue. The residue is triturated with 15% ethyl acetate/heptane to give the title product as a white solid, 4.25 g, 69.2% yield, mp 96°-97° C., characterized by ¹HNMR and MS analyses.

### EXAMPLE 2

Preparation of 3′-Bromo-2,3,4,6′-tetramethoxy-2′, 6-dimethylbenzophenone

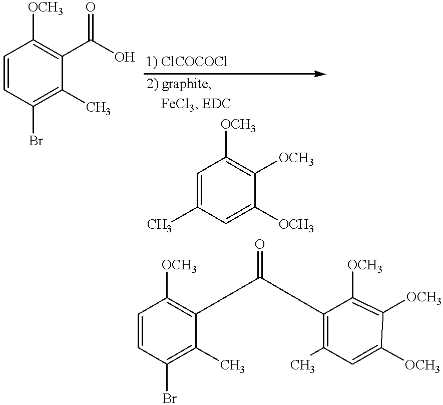

A slurry of 3-bromo-6-methoxy-2-methylbenzoic acid (7.35 g, 0.03 mol) in 1,2-dichloroethane (EDC) is treated with oxalyl chloride (4.18 g, 0.033 mol) at room temperature over a 15 minute period, heated to 70° C. for 2 hours, cooled to room temperature, treated with 3,4,5-trimethoxytoluene (5.5 g, 0.03 mol), anhydrous FeCl₃ (16 mg, 0.33 mol %), and graphite (250 mg), heated at reflux temperature for 3 hours (reaction complete by GLC analysis) and filtered. The filtrate is washed with saturated NaHCO₃ solution and concentrated in vacuo to give a residue. The residue is triturated with 15% ethyl acetate/heptane to give the title product as a pale gray solid, 8.8 g, 71.7% yield, mp 96°-97° C.

### EXAMPLE 3

Preparation of 3′-Bromo-2,3,4,6′-tetramethoxy-2′,6-dimethylbenzophenone

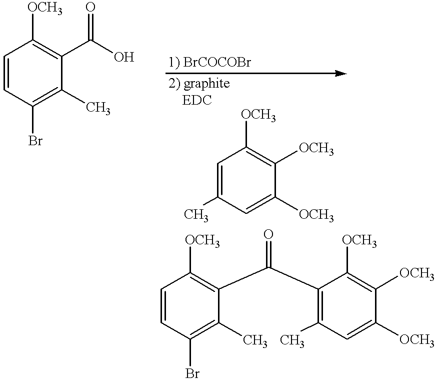

A slurry of 3-bromo-6-methoxy-2-methylbenzoic acid (4.9 g, 0.02 mol) in 1,2-dichloroethane (EDC) is treated with oxalyl bromide (4.75 g, 0.022 mol) at room temperature over a 15 minute period, heated to 50° C. for 2 hours, cooled to room temperature and concentrated in vacuo to give the corresponding acid bromide. This crude acid bromide is dissolved in EDC, treated with 3,4,5-trimethoxytoluene (1.82 g, 0.01 mol) and graphite (1.0 g), heated at reflux temperature for 3 hours [reaction is complete by TLC (silica gel, 25% ethyl acetate/heptane)], cooled to room temperature and filtered. The filtrate is washed with saturated NaHCO₃ solution and concentrated in vacuo to give a brown oil residue. The residue is purified by flash column chromatography on silica gel, packed and eluted with 15% ethyl acetate/heptane, to give the title product as a white crystalline solid, 2.2 g, 53.8% yield, mp 97°-98° C., characterized by ¹HNMR and MS analyses.

### EXAMPLE 4

Preparation of 4′-Chloro-2,3,4-trimethoxy-5-methylbenzophenone

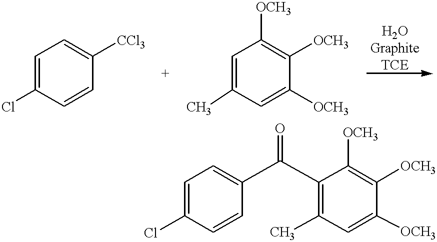

A mixture of p-chlorobenzotrichloride (6.9 g, 0.03 mol), 3,4,5-trimethoxytoluene (6.6, g, 0.036 mol), and graphite (1.5 g) in 1,1,2,2-tetrachloroethane (TCE) is heated to 100° C., treated with water (0.6 g, 0.036 mol), heated to reflux temperature for 1.5 hours, treated with additional water (0.48 g, 0.026 mol), heated for another 2.5 hours at reflux temperature, cooled to room temperature and filtered. The filtrate is washed with aqueous NaHCO₃ solution and concentrated in vacuo to give an oil residue. The residue which is purified by flash column chromatography on silica gel, packed and eluted with 15% Ethyl acetate/heptane, to give the title product as a white crystalline solid, 6.2 g, 64.4% yield, mp 99.5°-100.0° C., characterized by ¹HNMR and MS analyses.

### EXAMPLE 5

Preparation of 2,3,4-Trimethoxy-6-methyl-benzophenone

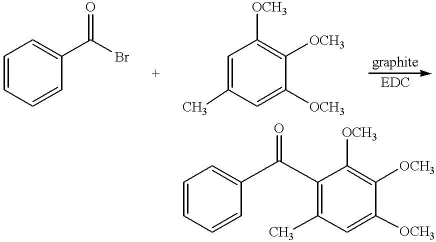

A mixture of benzoyl bromide (2.8 g, 0.015 mol), 3,4,5-trimethoxytoluene (1.8 g, 0.01 mol) and graphite (1.0 g) in 1,2-dichloroethane (EDC) is heated at reflux temperature for 9 hours, cooled to room temperature and filtered. The filtrate is washed with saturated NaHCO₃ solution and concentrated in vacuo to give a residue. The residue is triturated with 15% ethyl acetate/heptane to give the title product as a pale yellow solid, 1.2 g, 42% yield, mp 88°-89° C., characterized by ¹HNMR and MS analyses.

### EXAMPLE 6

Preparation of 4′-Chloro-3,4-dimethoxybenzophenone

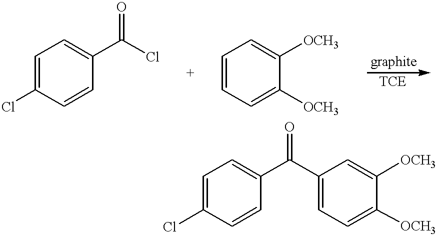

A slurry of p-chlorobenzoyl chloride (5.25 g, 0.03 mol), veratrole (4.15 g, 0.03 mol) and graphite (1.5 g) in TCE is heated at reflux temperature for 8 hours, cooled to room temperature and filtered. The filtrate is washed with saturated NaHCO₃ solution and concentrated in vacuo to give an oil residue. The residue is triturated with 15% ethyl acetate/heptane to give the title product as a pale yellow solid, 6.75 g, 81.3% yield, mp 110°-112° C., characterized by ¹HNMR analysis.

### EXAMPLE 7

Preparation of 4′-Chloro-3,4-dimethoxybenzophenone

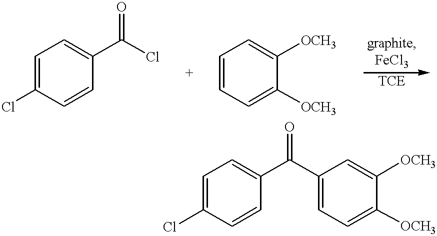

A slurry of p-chlorobenzoyl chloride (5.25 g, 0.03 mol), veratrole (4.56 g, 0.033 mol), anhydrous FeCl₃ (16 1 mg, 0.33 mol %) and graphite (250 mg) in TCE is heated at reflux temperature for 3 hours, cooled to room temperature and filtered. The filtrate is washed with saturated NaHCO₃ solution and concentrated in vacuo to give an oil residue. The residue is triturated with 15% ethyl acetate/heptane to give the title product as a white solid, 7.0 g, 84.3% yield, mp 110°-112° C.

### EXAMPLE 8

Preparation of 4′-Chloro-3,4-dimethoxybenzophenone

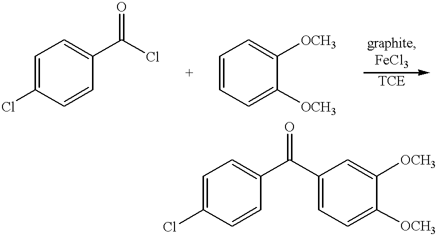

A slurry of p-chlorobenzoyl chloride (5.25 g, 0.03 mol), veratrole (4.56 g, 0.033 mol), FeCl₃ (8 mg, 0.16 mol %) and graphite (250 mg) in TCE is heated at reflux temperature for 8 hours and filtered. The filtrate is washed with saturated NaHCO₃ solution and concentrated in vacuo to give an oil residue. The residue is triturated with 15% ethyl acetate/heptane to give the title product as a white solid, 7.0 g, 84.3% yield mp 110.5°-111.5° C.

### EXAMPLE 9

Preparation of 4′-Chloro-3,4-dimethoxybenzophenone

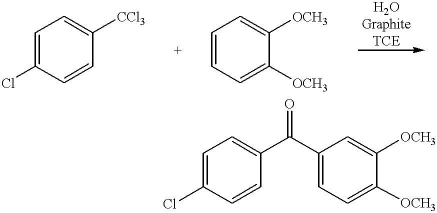

A mixture of p-chlorobenzotrichloride (6.9 g, 0.03 mol), veratrole (5.0, g, 0.036 mol), and graphite (1.0 g) in TCE is heated to 100° C., treated with water (0.6 g, 0.036 mol), heated to reflux temperature for 1.5 hours, treated with additional water (0.36 g, 0.02 mol) heated at reflux temperature for 4.5 hours, cooled to room temperature and filtered. The filtrate is washed with aqueous NaHCO₃ solution and concentrated in vacuo to give an oil residue. The residue is triturated with 15 ethyl acetate/heptane to give the title product as a white solid, 6.6 g, 79.5% yield, mp 110°-112° C.

### EXAMPLE 10

Preparation of 5-tert-Butyl-2′-chloro-2-methoxy-5′-nitrobenzophenone

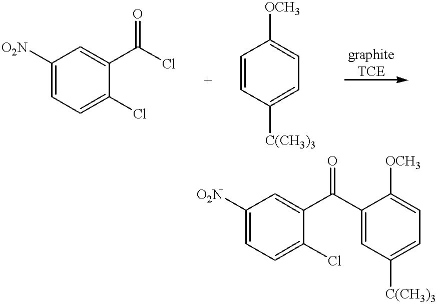

A slurry of 2-chloro-5-nitrobenzoyl chloride (2.2 g, 0.01 mol), p-tert-butylanisole (1.65 g, 0.01 mol), and graphite (0.5 g) in TCE is heated at reflux temperature for 2.5 hours, cooled to room temperature and filtered. The filtrate is washed with saturated NaHCO₃ solution and concentrated in vacuo to give an oil residue. The residue is triturated with 15% ethyl acetate/heptane to give the title product as a white solid, 2.0 g, 57.6% yield, mp 136°-139° C., characterized by ¹HNMR and MS analyses.

Using essentially the same procedure described hereinabove and substituting 1,2-dichloroethane as solvent, the title product is obtained as a white solid in 79.6% yield, mp 136°-139° C.

### EXAMPLE 11

Preparation of 5-tert-Butyl-2′-chloro-2-methoxy-5′-nitrobenzophenone

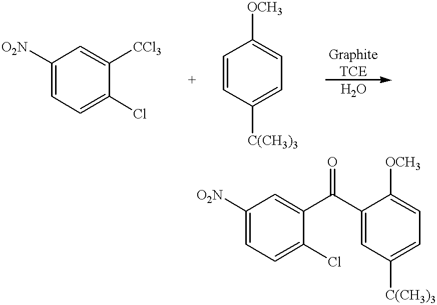

A mixture of 2-chloro-5-nitrobenzotrichloride, 2.75 g, 0.01 mol), p-tert-butylanisole (1.65g, 0.01 mol), and graphite (0.5 g) in TCE is heated to 100° C., treated with water (0.2 g, 0.011 mol), heated to reflux temperature for 2 hours, treated with additional water (0.2 g, 0.011 mol), heated at reflux temperature for 4 hours, cooled to room temperature and filtered. The filtrate is washed with aqueous NaHCO₃ solution and concentrated in vacuo to give an oil residue. The residue is triturated with 15% ethyl acetate/heptane to give the title product as a white solid, 1.75 g, 50.4% yield, characterized by TLC and ¹HNMR analyses.

### EXAMPLE 12

Preparation of 2′-Chloro-2,3,4-trimethoxy-5-methyl-5′-nitrobenzophenone

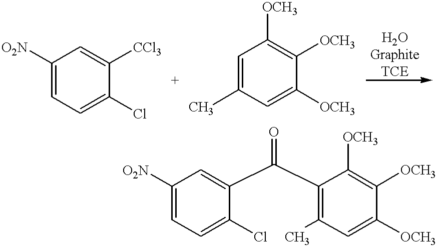

A mixture of 2-chloro-5-nitrobenzotrichloride (5.5 g, 0.02 mol), 3,4,5-trimethoxytoluene (3.65g, 0.02 mol), and graphite (1.5 g) in TCE is heated to 100° C., treated with water (0.36 g, 0.02 mol), heated at reflux temperature for 2 hours, treated with additional water (0.36 g, 0.02 mol) and heated at reflux temperature for 3 hours, cooled to room temperature and filtered. The filtrate is washed with aqueous NaHCO₃ solution and concentrated in vacuo to give an oil residue. The residue is purified by flash column chromatography on silica gel, packed and eluted with 15% ethyl acetate/heptane to give the title product as an oil which solidified on standing, 1.4 g, 19% yield, characterized by ¹HNMR and MS analyses.

### EXAMPLE 13

Preparation of 3,4-Dimethoxy-4′-nitrobenzophenone

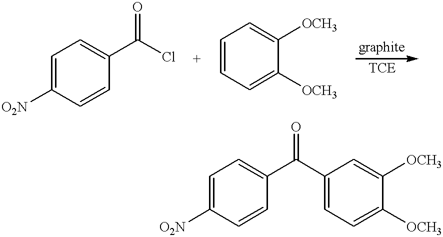

A slurry of p-nitrobenzoyl chloride (5.57 g, 0.03 mol), veratrole (4.15 g, 0.03 mol), and graphite (1.5 g) in TCE is heated at reflux temperature for 20 hours, cooled to room temperature and filtered. The filtrate is washed with saturated NaHCO₃ solution and concentrated in vacuo to give an oil residue. The residue is triturated with 15% ethyl acetate/heptane to give a yellow solid. The solid is mixed with 15% ethyl acetate in hexane and heated at reflux temperature for 15 min., cooled and filtered to give the title product as a yellow solid, 4.4 g, 51% yield, mp 160°-162° C.

### EXAMPLE 14

Preparation of 3,4-Dimethoxy-4′-nitrobenzophenone

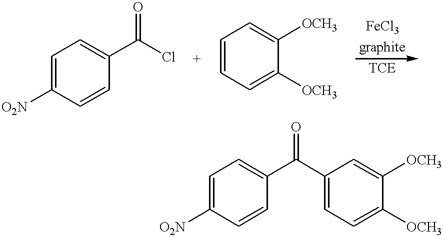

A slurry of p-nitrobenzoyl chloride (5.57 g, 0.03 mol), veratrole (4.15 g, 0.03 mol), anhydrous FeCl₃ (16 mg, 0.33 mol %), and graphite (250 mg) in TCE is heated at reflux temperature for 20 hours, cooled to room temperature and filtered. The filtrate is washed with saturated NaHCO₃ solution and concentrated in vacuo to give an oil residue. The residue is triturated with 15% ethyl acetate/heptane to give a yellow solid. The solid is dispersed in ethyl acetate, heated at reflux temperature for 15 min., cooled and filtered to give the title product as a yellow solid, 5.3g, 61.5 yield, mp 162°-167° C.

### EXAMPLE 15

Preparation of 5-tert-Butyl-2′-chloro-2-methoxy-4′-nitrobenzophenone

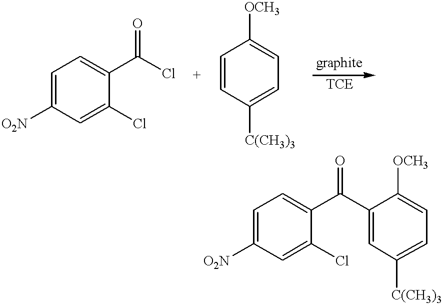

A mixture of 2-chloro-4-nitrobenzoyl chloride (7.92 g, 0.036 mol), p-tert-butylanisole (4.93 g, 0.03 mol), and graphite (1.0 g) TCE is heated at reflux temperature for 5 hours, cooled to room temperature and filtered. The filtercake is washed with TCE. The combined filtrates are washed with saturated NaHCO₃ solution and concentrated in vacuo to give an oil residue. The residue is purified by flash column chromatography on silica gel, packed and eluted with 15% ethyl acetate/heptane and by crystallization from 5% ethyl acetate/heptane, to give the title product as a white solid, 5.83g, 55.9% yield, mp 84°-85° C., characterized by ¹HNMR and MS analyses.

### EXAMPLE 16

Preparation of 5-tert-Butyl-2′-chloro-2-methoxy-4′-nitrobenzophenone

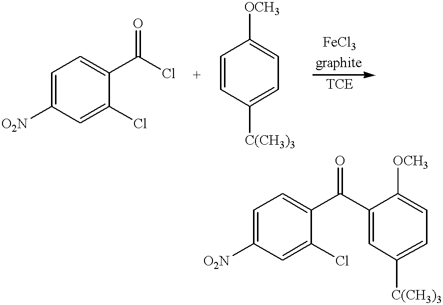

A mixture of 2-chloro-4-nitrobenzoyl chloride (7.92 g, 0.036 mol), p-tert-butylanisole (4.93 g, 0.03 mol), anhydrous FeCl₃ (16 mg, 0.33 mol %) and graphite (250 mg) in TCE is heated at reflux temperature for 2 hours, cooled to room temperature and filtered. The filtercake is washed with TCE. The combined filtrates are washed with saturated NaHCO₃ solution and concentrated in vacuo to give an oil residue. The residue is purified by flash column chromatography on silica gel, packed and eluted with 15% ethyl acetate/heptane, and by crystallization from 5% ethyl acetate/heptane to give the title product as a white solid, 5.7 g, 54.6% yield, mp 82.5°-83.5° C. characterized by ¹HNMR and MS analyses.

### EXAMPLE 17

Preparation of 2′, 6′-Difluoro-3,4-dimethoxybenzophenone

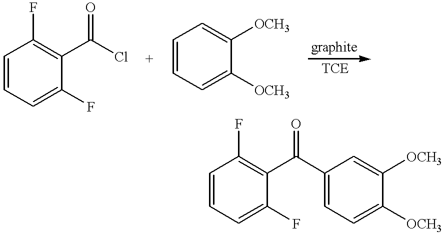

A slurry of 2,6-difluorobenzoyl chloride (5.3 g, 0.03 mol), veratrole (4.15 g, 0.03 mol), and graphite (1.5 g) in TCE is heated at reflux temperature for 1.5 hours, cooled to room temperature and filtered. The filtrate is washed with saturated NaHCO₃ solution and concentrated in vacuo to give an oil residue. The residue is triturated with 15% ethyl acetate/heptane to give the title product as a white solid, 6.7 g, 77.7% yield, mp 100.5°-101.0° C.

### EXAMPLE 18

Preparation of 2′, 6′-Difluoro-3,4-dimethoxybenzophenone

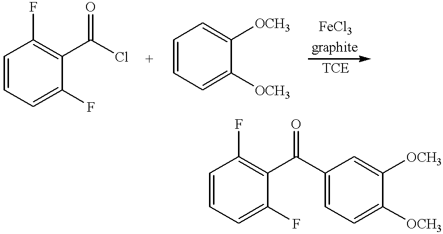

A slurry of 2,6-difluorobenzoyl chloride (5.3 g, 0.03 mol), veratrole (4.15 g, 0.03 mol), anhydrous FeCl₃ (16 mg, 0.33 molt), and graphite (250 mg) in TCE is heated at reflux temperature for 2.5 hours, cooled to room temperature and filtered. The filtrate is washed with saturated NaHCO₃ solution and concentrated in vacuo to give an oil residue. The residue is triturated with 15% ethyl acetate/heptane to give a brown solid. The solid is crystallized from 15% ethyl acetate/heptane to give the title product as a pale yellow solid, 3.9 g, 46.7% yield, mp 100°-101° C.

I claim:

1. A process for the preparation of a compound of formula I 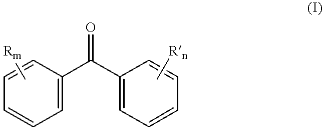
wherein m and n are each independently 0 or an integer of 1, 2, 3, 4 or 5;
R is halogen, C₁-C₆alkyl, C₁-C₆haloalkyl, C₁-C₆-alkoxy,C₁-C₆alkoxyalkyl, CO₂R₁, S(O)ₚR₂, NR₃R₄, NO₂ or CN;
R′ is C₁-C₆alkyl, C₁-C₆alkoxy, C₁-C₆alkoxyalkyl, or NR₅R₆;
R₁, R₂, R₃, R₄, R₅ and R₆ are each independently C₁-C₆ alkyl; and
p is 0 or an integer of 1 or 2
which process comprises reacting a compound of formula II 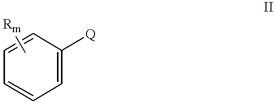
wherein Q is CX₃ or COX; X is Cl or Br; and R and m are as described hereinabove with at least one molar equivalent of a compound of formula III 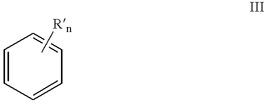
wherein R′ and n are as described hereinabove in the presence of graphite and an inert solvent, optionally in the presence of FeCl₃, and when Q is CX₃ in the presence of at least one molar equivalent of water.

2. The process according to claim 1 wherein the solvent is a halogenated aliphatic hydrocarbon or a halogenated aromatic hydrocarbon.

3. The process according to claim 2 wherein the solvent is a halogenated aliphatic hydrocarbon.

4. The process according to claim 1 having a formula II compound wherein Q is COX.

5. The process according to claim 1 having a formula II compound wherein Q is CX₃.

6. The process according to claim 1 wherein FeCl₃ is present.

7. The process according to claim 1 wherein the graphite is present at about 10 g/mole of formula III compound to 200 g/mole of formula III compound.

8. The process according to claim 6 wherein the FeCl₃ is present at about 0.001 mole % to 1.0 mole %.

9. The process according to claim 5 wherein water is present at about 1.0 molar equivalent to 3 molar equivalents.

10. A process for the preparation of a fungicidal compound of formula IV 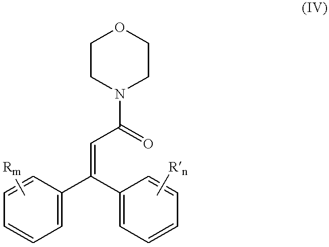
wherein m and n are each independently 0 or an integer of 1, 2, 3, 4 or 5;
R is halogen, C₁-C₆alkyl, C₁-C₆haloalkyl, C₁-C₆alkoxy, C₁-C₆alkoxyalkyl, CO₂R₁, S(O)ₚR₂, NR₃R₄, NO₂ or CN;
R′ is C₁-C₆alkyl, C₁-C₆alkoxy, C₁-C₆alkoxyalkyl, or NR₅R₆;
R₁, R₂, R₃, R₄, R₅ and R₆ are each independently C₁-C₆alkyl; and
p is 0 or an integer of 1 or 2
which process comprises reacting a compound of formula II 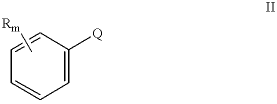
wherein Q is CX₃ or COX; X is Cl or Br; and R and m are as described hereinabove with at least one molar equivalent of a compound of formula III 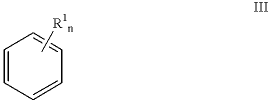
wherein R′ and n are as described hereinabove in the presence of graphite and an inert solvent, optionally in the presence of FeCl₃, and when Q is CX₃ in the presence of at least one molar equivalent of water to form a compound of formula I 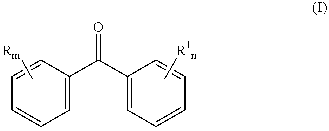
wherein m, n, R and R′ are as described hereinabove, and reacting said formula I compound with N-acetylmorpholine in the presence of sodium tert-alkoxide, optionally in the presence of a solvent, to give the desired fungicidal formula IV product.
